(19) United States
(12) Patent Application Publication
(10) Pub. No.: US 2011/0212152 A1
(43) Pub. Date: Sep. 1, 2011

(54) MODIFIED ANTI-MICROBIAL SURFACES, DEVICES AND METHODS

(76) Inventors: Valerio Ditizio, Toronto (CA); Frank DiCosmo, Richmond Hill (CA)

(21) Appl. No.: 12/982,178

(22) Filed: Dec. 30, 2010

Related U.S. Application Data
(63) Continuation-in-part of application No. 10/468,438, filed on Jan. 5, 2004.
(60) Provisional application No. 60/271,702, filed on Feb. 28, 2001.

(51) Int. Cl. A61K 9/00; A61K 47/48; A61P 31/00; B05D 3/06; C25D 5/48

(52) U.S. Cl. 424/422; 424/78.17; 427/2.1; 427/2.25; 427/2.29; 205/220

(57) ABSTRACT

Methods for making modified surfaces and in particular, surfaces that may be lubricious and may be further treated to be anti-microbial are disclosed. Devices comprising modified surfaces prepared by the methods are also disclosed. An exemplary method comprises incubating a photo-initiator-coated substrate in an aqueous monomer solution that is capable of free radical polymerization, exposing the incubating substrate to ultraviolet (UV) light creating a modified surface on the substrate. An anti-microbial agent may be added to the modified surface.

This application is a continuation-in-part of U.S. Ser. No. 10/468,438, which is a national stage entry of PCT/CA02/00246 filed Feb. 26, 2002, claiming priority from U.S. provisional application No. 60/271,702 filed Feb. 28, 2001, the contents of all of which are hereby incorporated herein by reference.

### FIELD OF THE INVENTION

The present invention relates to methods for making modified surfaces and in particular, surfaces that may be lubricious and may be further treated to be anti-microbial. Specifically, the present invention is directed to methods for the modification of the surfaces of a variety of substrates, including the surfaces of polymeric or metallic substrates, with polymer coatings that may be further treated to be anti-microbial.

### BACKGROUND

Throughout this application, various references are cited in parentheses to describe more fully the state of the art. The disclosure of these references is hereby incorporated by reference into the present disclosure.

The use of implanted medical devices is a vital component of current clinical practice. However, complications may arise from their use. Common complications are the physical trauma to the patient's tissues resulting from insertion and continued use of the device, as well as the potential for the device to serve as a focus for microbial contamination and thus, a possible source for microbial infection of the patient. In fact, these complications are often associated since the placement of a medical device, such as a urethral catheter or ureteral stent, may cause tearing and bleeding of delicate tissues thereby creating an opportunity for infection through microbial contamination of the device or through subsequent migration of microbes along the device's surface. It has therefore been an object to develop better quality indwelling biomedical devices made from materials that provide for clinical advantages to a patient.

In response to the problem of insertion-related trauma, polymeric medical devices have been coated with various hydrophilic polymers to produce a more low friction or lubricious coating on the device. The coated devices have high friction surfaces when dry, but upon wetting, the device becomes slippery and can be more readily inserted into veins, arteries, and other passageways causing minimal tissue damage. However, the methods to apply hydrophilic-coating processes as well as the coatings themselves possess several distinct disadvantages, any one of which can significantly diminish the value of the end product. First, and perhaps foremost, is the inability to produce a permanent lubricious coating, as many coatings will erode after only a limited exposure to an aqueous environment (see Ikada, Y. and Uyama, Y., Surface Grafting. In Lubricating Polymer Surfaces. Technomic, Lancaster, Pa., 1993, pp. 111-137). Also, most of the current coating processes are resource-intensive procedures since they consist of at least two steps that require multiple compounds and organic solvents to produce the lubricious layer (see U.S. Pat. No. 4,585,666, Lambert, 1986; U.S. Pat. No. 5,662,960, Hostettler, F., Rhum, D., Forman, M. R., Helmus, M. N., Ding, N., 1997; U.S. Pat. No. 6,306,176, Whitbourne, R. J., 2001). Finally, many processes are incompatible with the use of various bio-active agents since they involve the use of organic solvents or a high temperature curing step (see U.S. Pat. No. 5,160,790, Elton, R., 1992; U.S. Pat. No. 5,620,738, Fan, Y. L., Marlin, L., Bouldin, L. M., and Marino, L. M., 1997). Even if the bio-active agent is compatible with other components of the coating, the capacity of the lubricious coating to allow for extended release of the agent is often limited because either the coating sloughs off or there is little inherent affinity between the coating and the agent.

Many of the polymers used to make medical devices are chemically inert requiring the introduction of reactive chemical groups to the polymeric surface in order to provide a more desirable bio-active surface. There are reports describing surface modification of polymers containing reactive functional groups introduced through the inclusion of derivatized monomers in the initial polymer formulation (see Saam, J. C.; Mettler, C. M.; Falender, J. R.; Dill, T. J., J. Appl. Polym. Sci. 1979, 24, 187; Cameron, G. G.; Chisholm, M. S., Polymer 1985, 26, 437; Holahan, A. T.; George, M. H.; Barrie, J. A., Makromol. Chem. Phys. 1994, 195, 2965). While this approach may yield adequate results, there are issues of convenience and the bulk properties of the polymer may be adversely affected. Similarly, surface modification using plasma discharge (see Okada, T. and Ikada, Y., Makromol. Chem. 1991, 192, 1705) and γ-irradiation (see Yang, J.-S, and Hsiue, G.-H., J. Appl. Polym. Sci. 1996, 61, 221) techniques as described for example in U.S. Pat. No. 5,885,566 may not always be practical because of the need for specialized equipment and the propensity for alteration of bulk material properties. Also, none of the above-mentioned procedures allow for precise spatial control of the surface modification reaction.

Surface graft polymerization using long-wave ultraviolet (UV) light has been shown to be an efficient and convenient method for modifying polymer surfaces with the added benefit of micro-regional control through the use of projection masks (see Inoue, H. and Kohama, S., J. Appl. Polym. Sci. 1984, 29, 877). One common strategy for surface photografting uses benzophenone and related molecules to abstract hydrogen atoms from the polymer surface, thereby creating surface-bound radicals capable of initiating graft polymerization of monomers in the vapour phase or in solution (see After, K.; Hult, A.; Ranby, B., J. Polym. Sci. A: Polym. Chem. 1988, 26, 2099; Ulbricht, M.; Riedel, M.; Marx, U., J. Membr. Sci. 1996, 120, 239). U.S. Pat. No. 6,248,811 discloses surface grafting of a coating polymer to a portion of a surface of a substrate using UV radiation. The resultant surface may be antibacterial and further inhibit or promote cell proliferation.

Attempts have also been made to add anti-microbial agent(s) to a surface modified polymer as is disclosed for example in U.S. Pat. No. 5,788,687 in which the anti-microbial agents acetohydroxamic acid and magnesium ammonium phosphate hexahydrate are released upon a change of pH from a polymer hydrogel that is coated onto a polymeric surface.

Silver is known to have general anti-microbial properties directed against a wide range of bacteria and fungi and has been used for many years in clinical settings and particularly on a wide range of medical devices which include coatings for catheters, cuffs, orthopedic implants, sutures, dental amalgams and wound dressings. As a coating on catheters silver has been demonstrated to reduce the incidence of infection associated with the use of such devices. Both silver alloy and silver oxide has been used to coat urinary catheters and are somewhat effective in preventing urinary tract infections (see Saint, S; Elmore, J. G.; Sullivan, S. D.; Emerson, S. S.; Koepsell, T. D.; Am. J. Med. 1998, 105:236-241). However, the use of silver as a prophylactic against infection in general, has not found widespread application because of problems associated with inadequately coating device surfaces, poor solubility of metallic silver and silver oxides, short half-life, rapid binding of silver ions and inactivation by proteins and light-mediated inactivation and discoloration, and slow release of silver ions from the metallic complex.

Thus, there is a need to develop a method to effectively modify the surface of various materials, which form the basis for clinically used medical devices, in a manner such that the surface is lubricious and can further be modified to have anti-microbial properties in a manner that obviates at least one problem with that of the prior art.

### SUMMARY OF THE INVENTION

The present invention provides methods for making various modified surfaces and furthermore, modified anti-microbial polymeric surfaces on polymeric, metallic or other materials. Specifically, the present invention provides methods for the modification of the surfaces with stable polymer coatings to make the surfaces, for example, more bio-compatible and lubricious.

Exemplary methods aim to modify the surfaces of a wide variety of materials such as for example silicone rubber that are used clinically in viva with polymer coatings treated with silver salts for the provision of anti-microbial surfaces in order to prevent, ameliorate and treat bacterial and fungal infections in humans and mammals. One skilled in the art would readily understand exemplary polymeric and other materials that can be modified in accordance with the present invention.

According to one embodiment, a hydrophilic polyacrylate-modified polymeric surface is provided. In a further embodiment, the acrylate coating of the polymeric surface is used to retain a silver component that is released in order to treat and help prevent bacterial and fungal infections. In still another embodiment, the acrylate-modified silicone surface has incorporated therein a silver component within a polyethylene oxide hydrogel capable of releasing silver. In one aspect of this embodiment, the silver component is provided encapsulated with liposomes that are provided within the polyethylene oxide hydrogel.

The silver component can take a variety of different formats. For example, the silver component can be a silver salt. One group of silver salts includes silver phosphate, silver citrate and silver lactate. Other suitable silver salts include but are not limited to silver acetate, silver benzoate, silver chloride, silver carbonate, silver iodide, silver iodate, silver nitrate, silver laurate, silver sulfadiazine, silver palmitate, silver salicylate, silver thiosulfate, and mixtures thereof.

In accordance with an aspect of the present invention is a method for making a modified surface on a polymeric material, the method comprising: incubating a photo-initiator-coated polymeric material with an aqueous monomer capable of free radical polymerization; and exposing the incubating polymeric material to UV light creating a modified surface on said polymeric material.

According to yet a further aspect of the present invention is a polymeric composite comprising: a polymeric body having a stable polyacrylate modified surface, said surface being hydrophilic, lubricious and anti-microbial.

According to a further aspect of the present invention is a polyacrylate coated polymer.

According to a further aspect of the present invention is an anti-microbial polyacrylate coated polymer having a silver component within the polyacrylate coating.

According to a further aspect of the present invention is an anti-microbial polyacrylate coated polymer having a silver component within the polyacrylate coating, wherein the silver component is released from the polyacrylate coating continuously over a period of time.

According to another aspect of the invention is a method for making a lubricious modified surface on a polymeric material, comprises; incubating a photo-initiator-coated polymeric material with an aqueous monomer capable of free radical polymerization; exposing the incubating polymeric material to UV light creating a modified surface on said polymeric material; rendering said modified surface lubricious.

According to a further aspect of the present invention is a method for making a lubricious anti-microbial modified surface on a polymeric material, the method comprising: incubating a photo-initiator-coated polymeric material with an aqueous monomer capable of free radical polymerization; exposing the incubating polymeric material to UV light creating a modified polymeric surface on said polymeric material; rendering said modified surface lubricious; and adding a silver agent to said lubricious modified polymeric surface.

According to another aspect of the present invention is a method for making a lubricious anti-microbial modified surface on a polymeric material, the method comprising: precoating a polymeric material with a photo-initiator; immersing the precoated polymeric material in an aqueous solution of vinyl carboxylic acid monomer and exposing the incubating polymeric material to UV light to create a modified non-lubricious polyacrylate surface on said polymeric material; ionizing said polyacrylate surface of said polymeric material by immersion in an aqueous base; saturating the polyacrylate surface with cations by immersion in an electrolyte solution; and providing silver to said cation-saturated polyacrylate surface.

The silver may be provided as a coating or incorporated within a hydrogel bonded to the acrylate modified polymeric material surface.

According to another aspect of the present invention is a method for making a modified surface on a substrate, the method comprising: incubating a photo-initiator-coated substrate in an aqueous monomer solution capable of free radical polymerization; exposing the incubating substrate to ultraviolet light creating a modified surface on the substrate; and rendering the modified surface lubricious.

According to a further aspect of the present invention is a method for making a modified surface on a substrate, the method comprising: coating the substrate with a photo-initiator; incubating the photo-initiator-coated substrate in an aqueous monomer solution capable of free radical polymerization; and exposing the incubating substrate to ultraviolet light creating a modified surface on the substrate.

According to a further aspect of the present invention is a method for making a modified surface on a substrate, the method comprising: coating the substrate with a photo-initiator; incubating the photo-initiator-coated substrate in an aqueous monomer solution capable of free radical polymerization; exposing the incubating substrate to ultraviolet light creating a modified surface on the substrate; and rendering the modified surface anti-microbial.

According to a further aspect of the present invention is a method for making a modified surface on a substrate, the method comprising: coating the substrate with a photo-initiator; incubating the photo-initiator-coated substrate in an aqueous monomer solution capable of free radical polymerization; exposing the incubating photo-initiator-coated substrate to ultraviolet light creating a modified surface on the substrate; and ionizing the modified surface in an aqueous base solution so that the modified surface is negatively charged, followed by incubation with a silver salt solution.

According to a further aspect of the present invention is a method for making a modified surface on a substrate, the method comprising: coating the substrate with a photo-initiator; incubating the photo-initiator-coated substrate in an aqueous monomer solution capable of free radical polymerization; exposing the incubating substrate to ultraviolet light creating a modified surface on the substrate; and coating the substrate with the modified surface with a polyethylene oxide hydrogel having a silver salt incorporated therein.

According to a further aspect of the present invention is a method for making a modified surface on a substrate, the method comprising: immersing a substrate in an alcoholic solution of a photo-initiator for a time sufficient for the photo-initiator to adhere to the surface of the substrate; incubating the photo-initiator-coated substrate in an aqueous monomer solution capable of free radical polymerization; subjecting the incubating substrate to ultraviolet light at a suitable wavelength to initiate polymerization of the monomer on the surface of the substrate, creating a modified surface on the substrate; and rendering the modified surface anti-microbial.

According yet another aspect of the present invention is an implantable medical device comprising a substrate with a modified surface prepared according to a method described herein.

### BRIEF DESCRIPTION OF THE DRAWINGS

The present invention will be further understood from the following description with reference to the Figures, in which:

FIG. 1(A) shows the amount of poly(acrylic acid [AA]) grafted onto poly(dimethylsiloxane) [PDMS] as a function of UVA irradiation time. Samples were initially coated with 10 mM p-benzoyl tert-butylperbenzoate (BPB). AA concentration in the BPB-saturated aqueous solution was 50 mg/mL (694 mM). FIG. 1(B) shows poly(AA) grafting extent with respect to initial monomer concentration. Samples were coated with 10 mM BPB and exposed to UVA light for 20 minutes.

FIG. 2 shows grafting yields of various polymers onto PDMS as a function of BPB pre-coating (100 mM) or BPB presence in the monomer solution. Monomer concentration was 694 mM and samples were exposed to UVA light for 20 minutes.

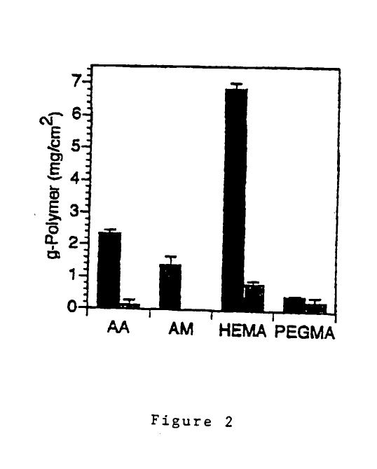

FIG. 3 shows the water contact angles of various types of surface modified silicone measured by the axisymmetric drop shape analysis technique. Samples were coated with 100 mM BPB and exposed to UVA light for 20 minutes unless otherwise noted.

FIG. 4 shows low and high resolution XPS spectra of (A) PDMS, (B) PDMS-g-poly(AA), (C) PDMS-g-poly(polyethylenglycol methacrylate [PEGMA]), (D) PDMS-g-poly(hydroxyethyl methacrylate [HEMA]) and (E) PDMS-g-poly(acrylamide [AM]). Samples were obtained from 100 mM BPB pre-coating and 20 min UVA exposure time.

FIG. 5 illustrates the anti-microbial activity of the polyacrylate-silver coating on silicone against Pseudomonas aeruginosa.

FIG. 6 illustrates the initial loading of silver salt on poly(AA)-modified catheters obtained by autoclaving in 150 mM silver lactate (Autoclaved) solution, or incubating overnight (O/N incub.) or incubation in 150 mM Nacl overnight, followed by a 2 hour incubation in 150 mM silver lactate solution (AgCl precip.).

FIG. 7 illustrates the extended anti-microbial activity of gelatin-poly(ethylene oxide) coatings against Pseudomonas aeruginosa and Staphylococcus aureus.

FIG. 8 illustrates the anti-microbial activity of the polyacrylate-silver coating on polyurethane stents against Pseudomonas aeruginosa and Staphylococcus aureus.

In the drawings, preferred embodiments of the invention are illustrated by way of example. It is to be expressly understood that the description and drawings are for the purpose of illustration and as an aid to understanding, and are not intended as a definition of the limits of the invention.

### DETAILED DESCRIPTION

Exemplary of an embodiment is a method for modifying the surface of a variety of organic or inorganic substrates with polymeric coatings containing various functional groups, in particular, polyacrylate coatings, that can be optionally, made lubricious and optionally, endowed with properties, such as anti-microbial activity. As such, the method may provide for an organic or inorganic substrate that has a stable hydrophilic and bio-compatible surface which can be further provided to possess some characteristics, for example, lubricity and anti-microbial properties. The method further provides an implantable medical device that has a stable hydrophilic and bio-compatible surface which can be further provided to possess optionally, the characteristics of lubricity and optionally, anti-microbial properties.

Suitable organic or inorganic substrates include, but are not limited to, polymers including natural or synthetic polymers, metals, metal alloys, metal oxides, quartz, ceramics, glasses, glass-ceramics, silicate materials, carbon materials such as graphite, or any combination thereof.

Suitable organic or inorganic substrates include those typically used in a variety of medical devices, including in-dwelling medical devices which are manufactured, for example, to replace a missing biological structure, support a damaged biological structure, or enhance an existing biological structure, and devices in general. Such medical devices include but are not limited to dressings, pins, clips, catheters, stents, implants, tubings, rods, prostheses, screws, plates, stents, endotracheal tubes, heart valves, dental implants and the like. The methods described herein can also be used to coat metal adornments such as metal body piercing materials (studs and the like). Similarly, other substrate surfaces that may be implanted in or in contact with a subject's body may be coated using the described methods.

One group of substrates that may be coated includes polymer substrates that can be surface modified and may include various types of polymeric substrates which would be readily understood by one skilled in the art, such as polyurethanes, polyamides, polyesters, polyethers, polyorganosiloxanes, polysulfones, polytetrafluoroethylene, polysiloxanes, Latex rubber, polyethylene, polyether ether ketone and the like.

Another group of substrates that may be coated includes metal substrates, including metal alloy substrates, such as but not limited to titanium, both pure titanium and/or a metallic titanium alloy containing one or more of chromium, nickel, aluminum, vanadium, cobalt, for example but not limited to TiAl₆V₄, TiAlV₄, TiAlFe_{2.5}. The metal substrate can also be fabricated of stainless steel such as implant grade material, e.g. V2A, V4A. Implant grade steel includes martensitic steel and austenitic steel, e.g. 316L and 316LVM. Cobalt-chromium alloys may also be coated.

The substrates may be suitably coated with a photo-initiator in manners known to those of ordinary skill in the art. For example, the photo-initiator may be coated onto the surface of selected organic or inorganic substrate by incubating the substrate in an alcoholic solution of photo-initiator for a time sufficient for the photo-initiator to adhere to the surface of the substrate.

Coating can be followed by air-drying of the photo-initiator-coated substrate.

According to the methods described herein, the substrates may be coated with polymeric coatings containing various functional groups so that the polymer coatings may be rendered, for example, hydrophilic, bio-compatible, and/or anti-microbial.

An exemplary method specifically involves forming a hydrophilic polymeric coating to the surface of a photo-initiator-coated organic or inorganic substrate through the use of UV radiation.

The method comprises an initial step of free radical-mediated graft polymerization of acrylic acid or various other acrylates on a photo-initiator-coated organic or inorganic substrate placed in an aqueous solution of monomer and exposed to UV light.

Exemplary of an embodiment, a polymer substrate may be initially coated with a photo-initiator to accept a polyacrylate coating. The polyacrylate coating may then be rendered lubricious and if further desired, provided with an anti-microbial agent such as but not limited to a silver agent.

Exemplary of another embodiment, a metal substrate may be initially coated with a photo-initiator to accept a polyacrylate coating. The polyacrylate coating may then be rendered lubricious and if further desired, provided with an anti-microbial agent such as but not limited to a silver agent. The metal substrate may be first optionally soaked for a suitable time period, for example, 5 minutes to 2 hours, in an organic solvent such as but not limited to acetone for de-greasing prior to grafting the substrate with the polyacrylate coating. The metal substrate may be de-greased in manners known to those of ordinary skill in the art.

Suitable photo-initiators include but are not limited to peresters, α-hydroxyketones, benzil ketals, benzoins and their derivatives and mixtures thereof. Specifically, suitable photo-initiators may be selected from 2,2-dimethoxy-2-phenyl-acetophenone (DPA), p-benzoyl tert-butylperbenzoate (BPB), benzophenone, tert-butyl peroxybenzoate and mixtures thereof. One skilled in art would readily understand the type of photo-initiator that can be used in the methods described herein.

Suitable monomers include but are not limited to monomers sensitive to the presence of free radicals, that is, monomers capable of free radical polymerization such as acrylic acid (AA), methacrylic acid, 2-carboxyethyl acrylate, 4-vinylbenzoic acid, itaconic acid, and mixtures thereof. In one embodiment, the monomer is acrylic acid.

A suitable wavelength for the UV radiation can be in the range of about 100 nm to about 400 nm. In some embodiments, suitable wavelength for the UV radiation can be in the range of about 200 nm to about 400 nm. In some embodiments, suitable wavelength for the UV radiation can be in the range of about 300 to about 400 nm.

One skilled in the art will appreciate that the level of grafting of the polymeric coating to the substrate surface may be controlled by adjustment of photo-initiator and monomer concentrations, as well as duration of irradiation. Suitable conditions can be readily determined using routine laboratory methods.

When working with silicone as the substrate polymer, grafting levels of nearly 1 mg/cm² may be achieved with as little as 2 minutes of irradiation time using aqueous AA (5 wt %).

In one embodiment, it is demonstrated that the photosensitive perester BPB leads to significant graft polymerization onto PDMS in a surface photografting reaction.

In other embodiments, it is further demonstrated that a BPB coating on PDMS can induce extensive graft polymerization of a number of hydrophilic monomers in an aqueous solution when exposed to 365 nm UV light.

When working with metal substrates, grafting levels of about 0.5-1 mg/cm² may be achieved with as little as 2 minutes of irradiation time using aqueous AA (5 wt %).

In an embodiment, modification of the surface of a metal substrate involves grafting a hydrophilic polyacrylate to the surface of the metal substrate through the use of long wavelength UV radiation (about 300 to about 400 nm) and a photo-initiator such as BPB.

In another embodiment, it is demonstrated that a benzophenone and tert-butyl peroxybenzoate coating on a stainless steel substrate can induce extensive graft polymerization of acrylic acid in an aqueous solution when exposed to 350 nm UV light.

In a further embodiment, a benzophenone and tert-butyl peroxybenzoate coating on a titanium foil can induce extensive graft polymerization of acrylic acid in an aqueous solution when exposed to 350 nm UV light when compared with a dip coating of a titanium foil with polyacrylic acid. The grafted polyacrylic acid coating is strongly bound to the underlying titanium substrate when compared with the one obtained from dip coating.

Without being constrained to any particular theory, the mechanism for modifying the surface of a metal substrate with the polymer coating may be explained as follows: free radicals are generated on the surface of the metal substrate through the activities of the adsorbed photo-initiators upon exposure to UV light. The photo-initiator-derived radicals are capable of initiating the polymerization of monomers present in the surrounding monomer solution. At some point during the polymerization reaction, the growing polymer chains bind to the metal surface through a chain termination reaction, thereby creating a covalent link between the polymer and the metal.

The polymeric coating produced as described above may be hydrophilic but not very lubricious. To render a surface lubricious, the polymeric coating grafted onto a substrate may be further ionized in an aqueous base solution having a pH of greater than about 8.0. Suitable aqueous bases for use include but are not limited to disodium tetraborate (borate buffer), sodium carbonate, hydroxides such as ammonium hydroxide, calcium hydroxide, sodium hydroxide, Tris(tris(hydroxymethyl)aminomethane) and mixtures thereof.

Ionization of the polymeric coating produces a negatively charged surface. The negatively charged surface may then be saturated with cations to prepare the surface for coating with an anti-microbial agent, such as a silver agent. Saturation can be effected by immersion in an appropriate electrolyte solution. Should a silver solution be applied directly to the ionized polyacrylate coating, surface lubricity may be lost. Therefore, saturation in a suitable cation-saturating electrolyte solution prior to application of a silver solution may be required to maintain surface lubricity.

Without being constrained to any particular theory, it is believed that the loss of lubricity may be explained by complex formation of ionized carboxyl groups in the polymeric coating with positively charged silver ions; assuming that the surface lubricity is the result of the mutual repulsion of the many ionized polymeric chains grafted onto the substrate surface. Thus, saturation with cations, for example by immersion in sodium lactate, results in a large excess of cations such as sodium ions present in the polymeric coating which compete for carboxyl binding sites and prevent all of the silver ions binding to and deactivating the polymeric coating. The inability of cations such as sodium ions to reduce lubricity of polyacrylate coatings may be due to the much stronger affinity of carboxylates for silver ion. Also, silver ions may complex with multiple carboxylate sites.

One skilled in the art would readily understand the type of cation-saturating electrolyte solution that can be used in the methods described herein. It is important to note however, that the nature of the counter anion of the cation saturating solution should be such that it forms a complex with the anti-microbial agent, for example silver ions, which is at least slightly soluble. Suitable cation-saturating electrolytes include, but not limited to sodium acetate, sodium lactate, sodium citrate, sodium benzoate, sodium salicylate, sodium thiosulfate, disodium phosphate, potassium acetate, potassium lactate, potassium citrate, potassium benzoate, potassium salicylate, potassium thiosulfate, dipotassium phosphate, and mixtures thereof. In some embodiments, it is demonstrated that sodium lactate works well as a cation-saturating electrolyte where silver is used as the anti-microbial agent because silver lactate is a relatively soluble salt.

If the anti-microbial agent to be used is a silver anti-microbial agent, a suitable silver agent may be a silver salt, for example silver phosphate, silver citrate, silver lactate and mixtures thereof. Other suitable silver salts include but are not limited to silver acetate, silver benzoate, silver chloride, silver carbonate, silver iodide, silver iodate, silver nitrate, silver laurate, silver sulfadiazine, silver palmitate and mixtures thereof. The silver agent may be also incorporated within a hydrogel by encapsulation or association with pharmaceutical carriers such as liposomes, micelles, microcapsules, microspheres, nanospheres and mixtures thereof, as is described in more detail below.

In an embodiment, a polymer substrate with ionized polyacrylic acid grafted on its surface is incubated in a selected silver component such as a silver salt solution to produce an anti-microbial surface that releases silver ions. The ionized polyacrylic acid modified polymer substrate is incubated in a concentrated solution of sodium lactate (about 0.1 M to about 1.0M) for about 10 to 60 minutes, in some instances, about 30 minutes. The materials are then transferred to a selected silver salt solution (for example, silver lactate) for about 5 to 120 seconds, in some instances, about 60 seconds, in order to produce an anti-microbial surface that retains silver ions and slowly releases them over an extended period.

In another embodiment, an acrylate-modified silicone surface to which a silver salt-containing liposome-gelatin-polyethylene oxide hydrogel is covalently attached to release silver ions for treating, ameliorating and/or preventing bacterial and fungal infections in humans and mammals is provided. Polyacrylate-coated materials are activated for covalent binding to gelatin-poly(ethylene oxide) hydrogels by initial immersion in a solution of carbodiimide. The adhesion of the hydrogel coating to a surface-modified silicone material was, for example, found to increase approximately fifty-fold relative to unmodified silicone.

The binding of silver salts and the covalent binding of gelatin-poly(ethylene oxide) hydrogel containing silver salts encapsulated within liposomes to the surface of poly(acrylic acid)-grafted silicone samples acts to provide a source of readily available silver ions for the treatment and prevention of bacterial and fungal infections in humans and mammals. The manufacture and use of silver salts encapsulated within liposomes is disclosed in the co-inventor's co-pending U.S. Patent Application Ser. No. 60/159,427 filed Oct. 14, 1999 (the entirety of which is herein incorporated by reference). The manufacture of liposome-poly(ethylene oxide)-gelatin hydrogel for use in the methods of the present invention is disclosed in co-owned U.S. Pat. No. 6,132,765 (the entirety of which is herein incorporated by reference). The adhesion of the hydrogel coating to the surface-modified silicone was found to increase approximately fifty-fold relative to unmodified silicone.

In a further embodiment, stainless steel needles with grafted polyacrylic acid coating can be loaded with silver by immersing the stainless steel needles with grafted polyacrylic acid in an alkaline solution (50 mM Tris) for 5 minutes to ionize the grafted polyacrylic acid, followed by incubating the ionized polyacrylic acid-modified stainless steel needles in an aqueous solution of silver acetate for 30 minutes.

In another embodiment, silver can be loaded onto titanium foils with grafted polyacrylic acid by first immersing the titanium foils with grafted polyacrylic acid in an alkaline solution (50 mM Tris) for 6 minutes to ionize the grafted polyacrylic acid, followed by incubating the ionized polyacrylic acid-modified titanium foils in an aqueous solution of silver acetate for 10 minutes.

Exemplary methods provide a surface-modified substrate that has use as an in-dwelling device for a variety of different applications, including medical applications. The methods are mild and efficiently modify surfaces of various substrates in an easy and reliable manner. Furthermore, such surfaces are also provided as lubricious in order to facilitate their in vivo use. The modified surfaces may also be made to be anti-bacterial and/or anti-fungal in order to ameliorate and/or prevent and minimize and bacterial and/or fungal infection that can further compromise an individual in whom the device is implanted.

### EXAMPLES

The examples are described for the purposes of illustration and are not intended to limit the scope of the invention.

Methods of synthetic chemistry, biochemistry, molecular biology and histology referred to but not explicitly described in this disclosure and examples are reported in the scientific literature and are well known to those skilled in the art.

### Example 1

### Preparation of Acrylate-Modified Silicone Surface

Pre-weighed silicone disks (approximately 0.7 cm in diameter, 0.2 cm in thickness) or cylindrical sections (approximately 0.5 cm in diameter, 1 cm in length) were incubated in a methanol solution of photo-initiator (BPB; or p-benzoyl benzoic acid, BBA) for 1 hour followed by air drying at about 40° C. for 2 hours. Samples were then suspended in vials containing 3 mL of aqueous monomer solution. When required, monomer solutions were saturated with BPB. The aqueous solubility of BPB was 4 μg/mL. All solutions were filtered through 0.22 μm pore filters prior to being flushed with nitrogen for 15 min. Vials were sealed with rubber septa and placed 2.5 cm beneath a pair of UVA bulbs (15 W ea.). Radiation intensity at the sample site was 3.8 mW/cm² as determined by UV actinometry (Zhang, J. Y.; Esrom, H.; Boyd, I. W., Appl. Surf Sci. 1999, 138-139, 315). After completion of the graft polymerization reaction, samples were briefly washed under running water with occasional scrubbing to remove any signs of adsorbed homopolymer. The remaining non-grafted material was removed by overnight incubation in 50% ethanol followed by a 4 hour incubation in water. Samples were dried in a 60° C. oven for 16 hours before their weights were determined using a microbalance. Quadruplicate samples of each treatment were analysed in all experiments.

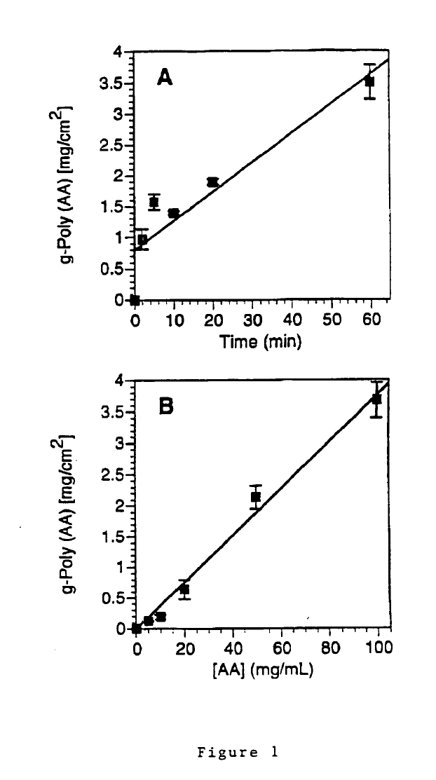

Free radical-mediated graft polymerization of acrylic acid (AA), acrylamide (AM), hydroxyethylmethacrylate (HEMA), and polyethylene glycol methacrylate (PEGMA) occurred on silicone surfaces when photo-initiator-coated samples were placed in aqueous solutions of monomer and exposed to UV light (FIG. 1). Grafting levels of nearly 1 mg/cm² were achieved with as little as 2 minutes of irradiation time (FIG. 1A) using aqueous acrylic acid (5 wt %). The level of grafting could be controlled by adjustment of photo-initiator and monomer concentrations, as well as duration of irradiation (FIG. 1B). Table 1 shows grafting of acrylic acid monomer to silicone polymer surface. The grafting of acrylic acid monomer is shown in milligrams (AA) per cm² of silicone polymer surface. In the presence of BPB, poly(AA) grafting onto silastic tubing, silicone Foley catheters and a silicone rubber disk was 3.0 mg/cm², 3.9 mg/cm² and 2.3 mg/cm², respectively.

TABLE 1
Grafting extent of selected samples with respect to
silicone source and photo-initiator identity.

| | Photo-initiator | Monomer | g-Polymer |
|---|---|---|---|
| Sample | (100 mM) | (694 mM) | (mg/cm²) |
| Silastic Tubing | BPB | AA | 3.0 ± 0.1 |
| Foley Catheter | BPB | AA | 3.9 ± 0.2 |
| Disk | BPB | AA | 2.3 ± 0.1 |
| Disk | BBA | AA | 0.05 ± 0.04 |

Surface Characterization.

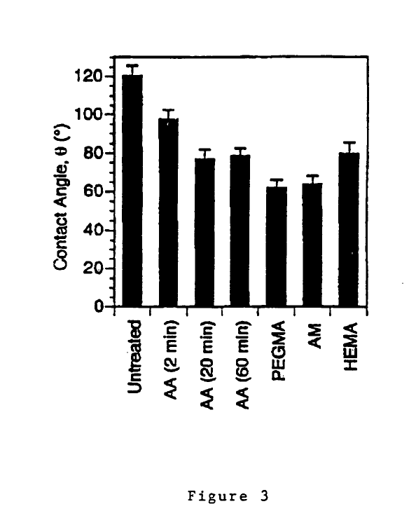

Silicone disk samples were kept in an atmosphere of high relative humidity for 24 hours prior to room temperature water contact angle measurements using axisymmetric drop shape analysis. Images of sessile water drops were digitized and contact angles determined by minimizing the difference between the proscribed drop volume and the drop volume calculated from the contact diameter of the drop in conjunction with the Laplace equation of capillarity (Moy, E.; Chenga, P.; Policova, Z.; Treppo, S.; Kwok, D.; Mack, D. R.; Sherman, P. M.; Neumann, A. W., Colloids Surfaces 1991, 58, 215) (FIG. 3). A total of 8 measurements on four different surfaces were performed for each treatment.

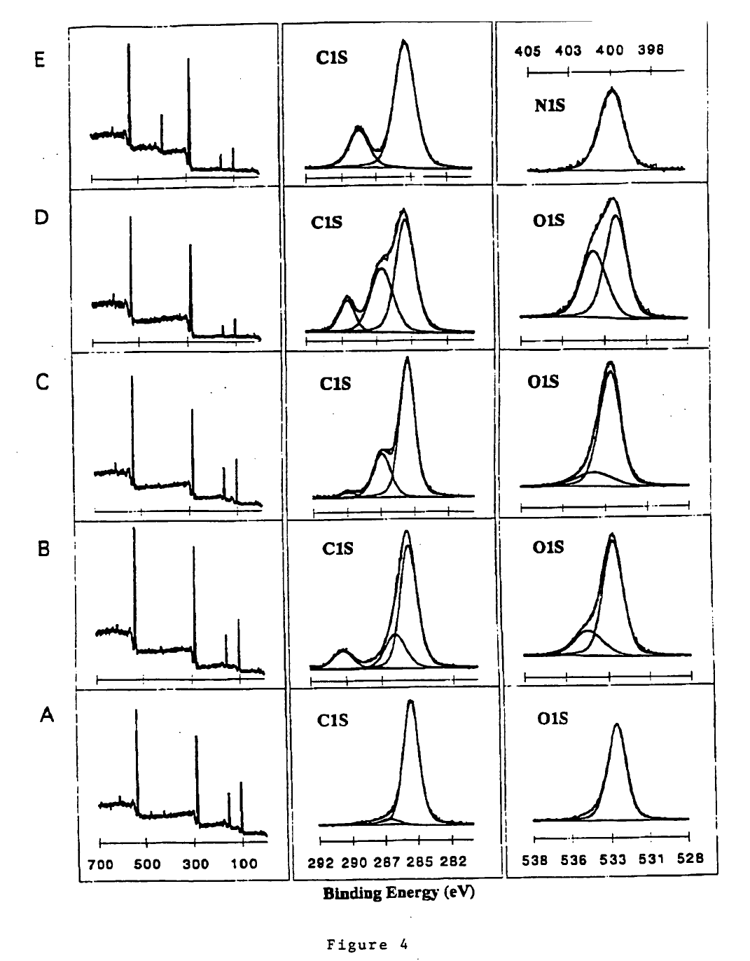

XPS spectra were recorded on a Leybold MAX 200×PS system utilizing an unmonochromatized Mg K x-ray source operating at 12 kV and 25 mA with a take-off angle of 90°. Energy range was calibrated against Cu 2p3/2 and Cu 3p at 932.7 eV and 75.1 eV, respectively, and scaled to place the main C peak at 285.0 eV. Binding energy determination and deconvolution of spectra were accomplished using the curve-fitting routines provided with the spectrometer. XPS spectra from two separate samples were recorded for each type of surface modification (FIG. 4).

### Example 2

### Preparation of Modified Poly-AA Lubricous Silicone Surface and the Lubricious Poly-AA-Silver Salt Modified Silicone Surface

Silicone Foley catheters coated with a lubricious poly(AA) coating containing silver ion was prepared in accordance with the following steps:

1. The silicone sheet or catheter portion was incubated in methanolic solution of photo-initiator (BPB; 20-250 mM, preferably 75 mM) for 1 hour at room temperature in the dark.

2. The catheters were removed from the BPB solution and air dried at room temperature for 1 hour.

3. The silicone material was placed in aqueous solution containing acrylate monomer (0.1-1.5 M, preferably 0.7 M of acrylic acid) to which was added a small amount of BPB (10-50 μg/mL; preferably 20 μg/mL).

4. The solution was bubbled with nitrogen while exposing the silicone material to 350 nm light (from 2 to 60 minutes, preferably 10 minutes.)

5. The surface modified silicone was placed in 50% ethanol for one hour followed by immersion in borate buffer (pH 9.0) overnight.

6. The lubricious surface-modified silicone was briefly washed in distilled water and placed in sodium lactate solution (200-1000 mM, preferably 500 mM) for a short period (2-120 minutes, preferably 20 minutes).

7. The surface modified silicone was placed in aqueous silver lactate solution (1-50 mM, preferably 10 mM) for 20 minutes. The modified silicone surface contains the silver salt bound to the acrylate coating and dissolved in the water associated with the coating.

The silicone material may alternatively be placed in aqueous silver lactate solution (2-200 mM, preferably 150 mM) and autoclaved at 15 psi for 20 minutes to yield silver lactate bound to the acrylate coating which is non-lubricious but anti-microbial.

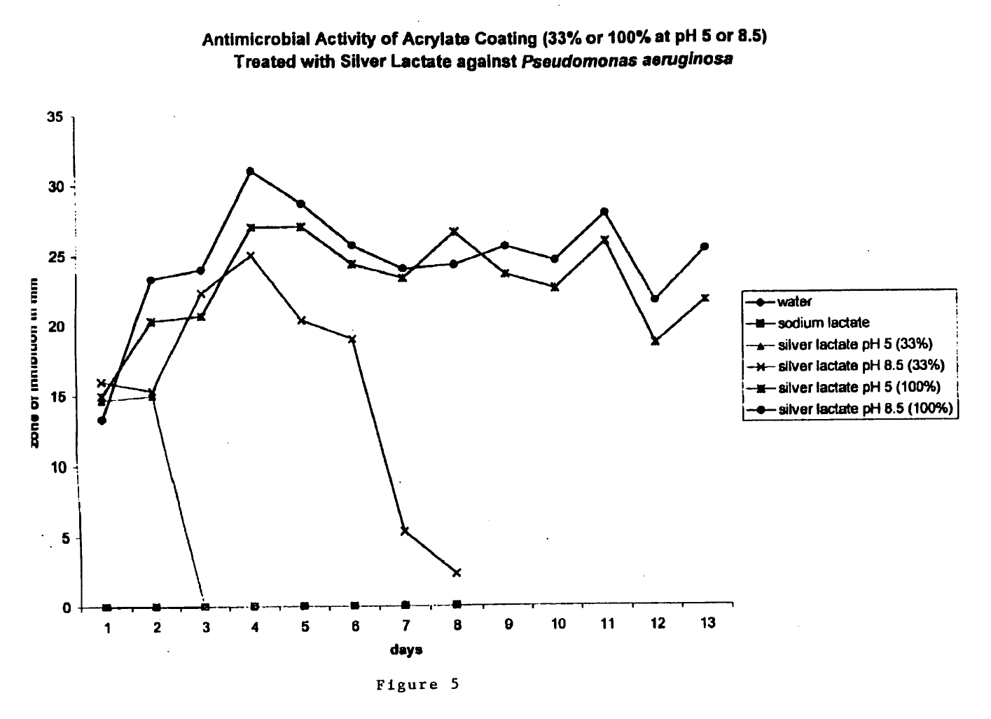

FIG. 5 illustrates the anti-microbial activity of the polyacrylate-silver coating on silicone against Pseudomonas aeruginosa. The greatest anti-microbial activities were produced by loading silver lactate in pH 5 and pH 8.5 solutions onto the surface of the silicone treated with 100% acrylate. Loading of silver salts at pH values higher or lower than those taught herein produce a surface showing substantial anti-microbial activity.

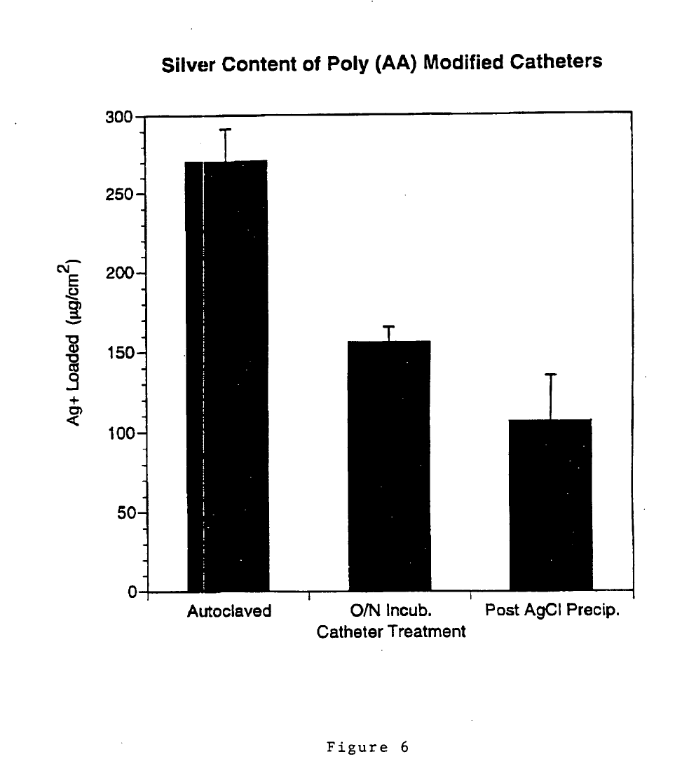

FIG. 6 shows the loading of a silver salt, silver lactate, on poly-acrylic acid modified catheters.

### Example 3

### Poly-AA Coated Silicone Sheets and Catheters with Attached Gelatin-Polyethylene Hydrogel Containing Silver Chloride

The poly(AA) coated silicone sheets and catheters with attached gelatin-polyethylene oxide hydrogel containing silver chloride were prepared as follows:

1. The relevant silicone material portion was incubated in methanolic solution of photo-initiator (BPB; 20-250 mM, preferably 75 mM) for 1 hour at room temperature in the dark.

2. The silicone was removed from the BPB solution and air dried at room temperature for 1 hour.

3. The silicone was placed in aqueous solution containing acrylate monomer (0.1-1.5 M, preferably 0.7 M of acrylic acid) to which was added a small amount of BPB (10-50 μg/mL; preferably 20 μg/mL).

4. The solution was bubbled with nitrogen while exposing silicone to 350 mm light (from 2 to 60 minutes, preferably 10 minutes).

5. The surface-modified silicone was placed in 50% ethanol and left overnight at room temperature with shaking.

6. The silicone was washed in distilled water for 4 hours.

7. The silicone was placed in carbodiimide solution (2-20 mg/mL preferably 5 mg/mL) for 10 minutes.

8. The catheter was removed and placed on rotating apparatus with the long axis rotating and the silicone sheets were placed on a flat surface.

9. A small volume of carbodiimide solution (preferably 10 μL/cm of catheter) was spread on the silicone surface.

10. The silver salt containing liposome hydrogel was prepared according to the following:

Composition:

| | | |
|---|---|---|
| DPPC (50 mg/ml) | 500 | mg |
| Cholesterol | 263.4 | mg |
| Vitamin E | 14.7 | mg |
| Silver lactate (150 mM) | 10.0 | ml |

The DPPC, cholesterol and vitamin E was dissolved in 10 ml chloroform and evaporated in a round bottle for at least 4 hours. Then 10 ml silver lactate (150 mM) was added to the above lipid film formulation and then heated to 45° C. until completely dispersed. This was then frozen in liquid nitrogen and thawed at 45° C. This was repeated 5 times. The mixture was extruded through a 100 nm filter and the filtrate collected. This was repeated 5 times. The silver lactate containing liposome mixture appears as a cream or yellowish cream suspension. The silver liposome suspension was then adjusted to pH 2 with HCl to convert the silver lactate not retained within liposomes to silver chloride to which was added 10% gelatin (w/v). The mixture was then heated to 45° C. until the gelatin was completely dissolved. Then 6-9% bis(nitrophenyl)polyethylene glycol 3400 (NP-PEG), preferably 9% was added and the mixture heated to 45° C. to dissolve the NP-PEG.

11. Silver chloride gel was added to the silicone surface (10-200 μL/cm of catheter, preferably 75 μL/cm), while rotating the catheter, or to a 10 cm×10 cm² silicone area was added 2-5 mL of silver chloride gel, preferably 1 mL and the gel was spread evenly over the surface.

12. Upon setting of the gel, the coated silicone was incubated at 4° C. for 10 minutes.

13. The coated silicone was then placed in borate buffer (200 mM; pH 9.0) for 1 hour.

14. The catheter was washed in saline solution for 2 hours replacing washing solution after 1 hour.

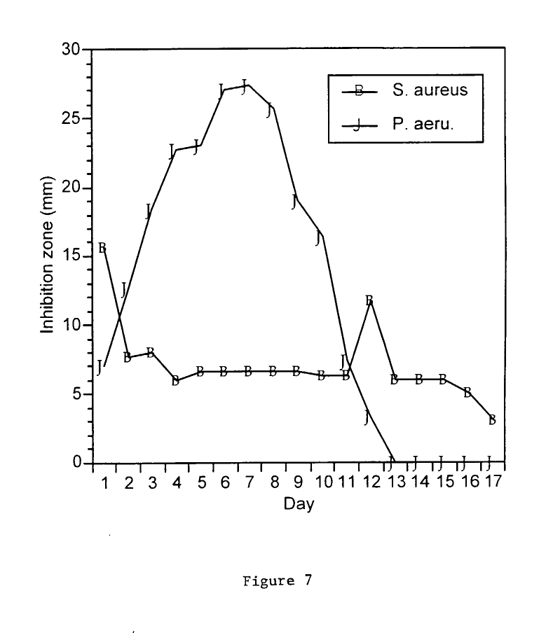

FIG. 7 illustrates the anti-microbial activity of hydrogel coatings; liposome-150 mM silver lactate hydrogel (1: liposome silver hydrogel) as compared to hydrogel without silver lactate (2: control hydrogel), hydrogel dipped in solution of 150 mM silver lactate (3: silver hydrogel), and filter paper dipped in 150 mM silver lactate (4: silver filter paper), against Pseudomonas aeruginosa and methicillin-resistant Staphylococcus aureus (MRSA). Note that the width of test hydrogel was 20 mm. The same test sample was used and transferred daily to a new agar plate. The value reported for zone of inhibition is the “actual” diameter of the zone from which 20 has been subtracted.

### Example 4

### Preparation of Polyurethane Ureteral Stents Coated with a Lubricious Poly (AA) Coating Containing Silver Ion

Poly (AA)-silver coated stents were prepared as follows:

1. The polyurethane stent was incubated in methanolic solution of photo-initiator (DPA; 20-250 mM, preferably 100 mM) for 1 minute at room temperature in the dark.

2. The stents were removed from the DPA solution and air dried at room temperature for 30 minutes.

3. The stents was placed in a DPA-saturated aqueous solution containing acrylate monomer (0.1-1.5 M, preferably 0.7 M of acrylic acid).

4. The solution was bubbled with nitrogen while exposing the stents to 350 nm light (from 1 to 20 minutes, preferably 2 minutes.)

5. The surface modified silicone was washed in 50% ethanol for 1 hour followed by immersion in borate buffer (pH 9.0) overnight.

6. The lubricious surface-modified polyurethane material was then briefly washed in distilled water and placed in sodium lactate solution (200-1000 mM, preferably 500 mM) for a short period (2-120 minutes, preferably 20 minutes).

7. The surface-modified stent was immersed in aqueous silver lactate solution (1-50 mM, preferably 10 mM) for 1 minute.

The modified polyurethane surface contains silver ion bound to the acrylate coating and dissolved in the water associated with the coating. The lubricity of the coating relative to unmodified polyurethane is illustrated in Table 2. Also, note the relatively non-lubricious nature of non-ionized poly(AA) coatings and ionized poly(AA) coatings that were not dipped in sodium lactate before immersion in silver solution.

TABLE 2
Lubricity of polyurethane stents (7 French) with and without
Polyacrylate (PAA) coatings.

| Sample | Friction (N) |
|---|---|
| Unmodifed | 3.3 ± 0.2 |
| PAA (ionized) | 0.45 ± 0.08 |
| PAA (non-ionized) | 3.8 ± 0.5 |
| | (10 mM) |
| PAA + Ag-Lac. | 2.2 ± 0.5 |
| | (10 mM) |
| PAA + Na-Lac. + Ag-Lac. | 0.46 ± 0.11 |

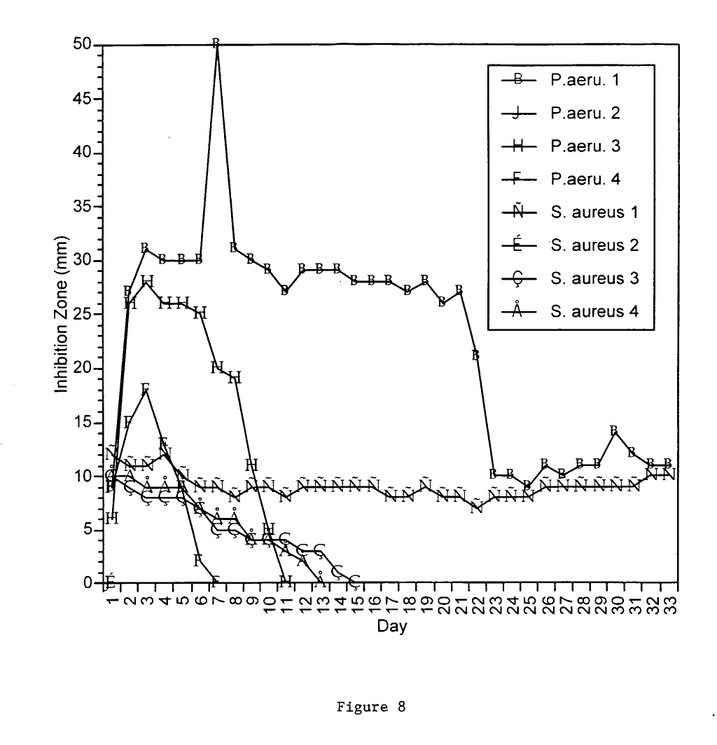

Anti-microbial activity of polyacrylate-silver coated stents is shown in FIG. 8. Growth inhibition zones were produced for extended periods with respect to both gram negative and positive species. Note that the width of stent pieces were approximately 2 mm. The same test sample was used and transferred daily to a new agar plate.

### Example 5

### Grafting of Polyacrylic Acid to Stainless Steel Needles

In situ photo-polymerization of acrylic acid with simultaneous grafting of polyacrylate thus formed to stainless steel needles (27 G—1.25 in.; Becton Dickinson) was accomplished as follows:

1. The stainless steel needles were cleaned/de-greased by being soaked in acetone for 10 minutes.

2. The de-greased stainless steel needles were immersed in an ethanol solution of photo-initiator (400 mM benzophenone+400 mM tert-butyl peroxybenzoate) for 10 minutes.

3. The photo-initiator-coated stainless steel needles were incubated in a 350 mM aqueous solution of acrylic acid and the solution was then purged with nitrogen to deoxygenate the solution for 6 minutes at ambient temperature.

4. The incubating photo-initiator-coated stainless steel needles in the aqueous solution of acrylic acid were exposed to UV light (350 nm) for 30 minutes at ambient temperature under nitrogen atmosphere to initiate polymerization of acrylic acid on the surfaces of the stainless steel needles, creating a polyacrylic acid coating, which was grafted to the surfaces of the stainless steel needles.

5. The polyacrylic acid grafted stainless steel needles were removed from the solution and then washed with deionized water for 60 seconds followed by washing with 50% ethanol for 60 seconds and drying in air at ambient temperature for 16 hours.

### Example 6

### Silver Loading of Polyacrylic Acid Grafted Stainless Steel Needles

The polyacrylic acid grafted stainless steel needles obtained from Example 5 were immersed in an alkaline solution (50 mM Tris; pH 9.0) for 5 minutes at ambient temperature.

2. The stainless steel needles with polyacrylic acid grafted surfaces prepared by the above step 1 of this example were transferred to and immersed in an aqueous solution containing 30 mM silver acetate for 30 minutes at ambient temperature.

3. The stainless steel needles with polyacrylic acid silver salt modified surfaces obtained from the above step 2 of this example were removed from the aqueous silver acetate solution, rinsed with deionized water for 60 seconds. Samples were air dried at ambient temperature for at least 16 hours.

Silver was extracted from the needle samples obtained from the above step 3 of this example by immersing the samples in extraction solution containing HNO₃ (1%)/NH₄OH (1%). Extraction was carried out for 16 hours at ambient temperature. The silver content of the extraction solution was measured using an atomic absorption spectrometer (Varian Spectra AA 50). Grafting level of approximately 78 μg/cm² of silver was achieved.

### Example 7

### Grafting of Polyacrylic Acid to Titanium Foils

In situ photo-polymerization of acrylic acid with simultaneous grafting to titanium foils (Aldrich; 99.7%) was accomplished as follows:

1. The titanium foils were cleaned/de-greased by being immersed in H₂SO₄ (98%)/H₂O₂ (30%) (2:1 v:v) for 30 minutes at 70° C.

2. The de-greased titanium foils were immersed in an isopropanol solution of photo-initiator (400 mM benzophenone+400 mM tert-butyl peroxybenzoate) for 10 minutes.

3. The photo-initiator-coated titanium foils were incubated in a 300 mM aqueous solution of acrylic acid (containing 1% by volume of the photo-initiators described in the above step 2 of this example in isopropanol) and the solution was then purged with nitrogen for 6 minutes to deoxygenate the solution at ambient temperature.

4. The incubating photo-initiator-coated titanium foils in the aqueous solution of acrylic acid (containing 1% by volume of the photo-initiators described in the above step 2 in isopropanol) were exposed to UV light (350 nm) for 20 minutes at ambient temperature under nitrogen atmosphere to initiate polymerization of acrylic acid on the surfaces of the titanium foils, creating a polyacrylic acid coating, which was grafted to the surfaces of the titanium foils.

5. The polyacrylic acid grafted titanium foils were removed from the solution and then washed with deionized water for 60 seconds followed by washing with 50% ethanol for 60 seconds and drying in air at ambient temperature for 16 hours.

### Example 8

### Dip Coating of Titanium Foils with Polyacrylic Acid

### Comparative Example

Titanium foils (Aldrich, 99.7%) were immersed in a 5% aqueous solution of polyacrylic acid (MW 450,000; Aldrich) for 5 minutes at ambient temperature.

### Example 9

### Silver-Binding Capacities of Grafted Versus Adsorbed Polyacrylic Acid on Titanium Foils

The silver-binding capacities of grafted samples prepared in Example 7 were compared to dip-coated samples obtained from Example 8. All samples were vigorously washed in running hot tap water for 30 seconds before the following silver-loading procedure was executed on either the sample prepared in Example 7 or the sample prepared in Example 8:

1. The sample was immersed in an alkaline solution (50 mM Tris, pH=9.0) for 6 minutes at ambient temperature.

2. The sample was transferred to and immersed in an aqueous solution containing 10 mM silver acetate for 10 minutes at ambient temperature.

3. The sample was rinsed with deionized water for 60 seconds and air dried.

After silver loading, the sample from Example 7 and the sample from Example 8 was each extracted with 35% nitric acid for 15 hours. The extraction solutions were analysed for silver content using an atomic absorption spectrometer (Varian Spectra AA-50) and the following results obtained:

Polyacrylic acid grafted to titanium foil: 20.5±0.7 μg of Ag/cm²

Polyacrylic acid adsorbed to titanium foil: 6.7±0.2 μg of Ag/cm²

The high silver-binding capacity of the sample with grafted polyacrylic acid suggests that the polymer coating was thicker/denser relative to the one obtained from dip coating. In addition, the ability of the thicker grafted coating to resist an aggressive wash step in running hot water suggests that the coating is strongly bound to the underlying titanium substrate.

Although preferred embodiments of the invention have been described herein in detail, it will be understood by those skilled in the art that variations may be made thereto without departing from the spirit of the invention or the scope of the appended claims.

What is claimed is:

1. A method for making a modified surface on a substrate comprising:
incubating a photo-initiator-coated substrate in an aqueous monomer solution capable of free radical polymerization;
exposing the incubating substrate to ultraviolet light creating a modified surface on the substrate; and
rendering the modified surface lubricious.

2. The method of claim 1, wherein the substrate comprises a polymer.

3. The method of claim 1, further comprising coating the substrate with the photo-initiator prior to the incubating.

4. The method of claim 3, wherein said coating comprises immersing the substrate in an alcoholic solution of the photo-initiator for a time sufficient for the photo-initiator to adhere to the surface of the substrate.

5. The method of claim 1, wherein the photo-initiator comprises a perester, an α-hydroxyketone, a benzil ketal, a benzoin, any derivative thereof or any mixture thereof.

6. The method of claim 1, wherein the photo-initiator comprises 2,2-dimethoxy-2-phenyl-acetophenone, p-benzoyl tert-butylperbenzoate, benzophenone or any mixture thereof.

7. The method of claim 1, wherein said incubating comprises immersing the photo-initiator-coated substrate in the aqueous monomer solution capable of free radical polymerization.

8. The method of claim 1, wherein the aqueous monomer solution additionally comprises a photo-initiator.

9. The method of claim 8, wherein the photo-initiator comprises a perester, an α-hydroxyketone, a benzil ketal, a benzoin, any derivative thereof or any mixture thereof.

10. The method of claim 8, wherein the photo-initiator comprises 2,2-dimethoxy-2-phenyl-acetophenone, p-benzoyl tert-butylperbenzoate, benzophenone or any mixture thereof.

11. The method of claim 1, wherein the monomer comprises acrylic acid, methacrylic acid, 2-carboxyethyl acrylate, 4-vinylbenzoic acid, itaconic acid, or any mixture thereof.

12. The method of claim 1, wherein said rendering comprises ionizing the modified surface of the substrate in an aqueous base solution so that the modified surface is negatively charged to provide a lubricious surface.

13. The method of claim 12, wherein the aqueous base has a pH of 8 or higher.

14. The method of claim 12, wherein the aqueous base comprises disodium tetraborate, sodium carbonate, ammonium hydroxide, calcium hydroxide, sodium hydroxide or any mixture thereof.

15. The method of claim 1, further comprising rendering the lubricious modified surface of the substrate anti-microbial.

16. The method of claim 15, wherein said rendering the lubricious modified surface of the substrate anti-microbial comprises incubating the substrate with the lubricious modified surface in an electrolyte solution to saturate the lubricious modified surface with cations followed by incubation with a silver salt solution.

17. The method of claim 16, wherein the silver salt comprises silver lactate, silver phosphate, silver citrate, silver acetate, silver benzoate, silver chloride, silver carbonate, silver iodide, silver iodate, silver nitrate, silver laurate, silver sulfadiazine, silver palmitate, silver salicylate, silver thiosulfate, or any mixture thereof.

18. The method of claim 16, wherein the electrolyte solution comprises sodium lactate, sodium acetate, sodium citrate, sodium benzoate, sodium salicylate, sodium thiosulfate, disodium phosphate, potassium acetate, potassium citrate, dipotassium phosphate, potassium benzoate, potassium salicylate, potassium thiosulfate or any mixture thereof.

19. The method of claim 15, wherein said rendering the lubricious modified surface of the substrate anti-microbial comprises coating the substrate with the lubricious modified surface with a polyethylene oxide hydrogel having a silver salt incorporated therein.

20. The method of claim 19, wherein the hydrogel is covalently bound to the lubricious modified surface.

21. The method of claim 19, wherein the hydrogel comprises a crosslinked matrix of gelatin and polyethylene oxide sequestering the silver salt.

22. The method of claim 21, wherein the silver salt comprises silver lactate, silver phosphate, silver citrate, silver acetate, silver benzoate, silver chloride, silver carbonate, silver iodide, silver iodate, silver nitrate, silver laurate, silver sulfadiazine, silver palmitate, silver salicylate, silver thiosulfate, or any mixture thereof.

23. The method of claim 1, wherein the substrate is provided as a medical device.

24. The method of claim 23, wherein the medical device is an implantable medical device.

25. The method of claim 24, wherein the implantable medical device comprises a dressing, a pin, a clip, a catheter, a stent, an implant, a tubing, a rod, a prosthesis, a screw, a plate, a stent, an endotracheal tube, a heart valve or a dental implant.

26. A method for making a modified surface on a substrate comprising:
coating the substrate with a photo-initiator;
incubating the photo-initiator-coated substrate in an aqueous monomer solution capable of free radical polymerization; and
exposing the incubating substrate to ultraviolet light creating a modified surface on the substrate.

27. The method of claim 26, further comprising rendering the modified surface anti-microbial.

28. The method of claim 26, wherein said rendering the modified surface anti-microbial comprising providing a silver agent to the modified surface.

29. The method of claim 28, wherein said providing a silver agent to the modified surface comprises ionizing the modified surface in an aqueous base solution so that the modified surface is negatively charged, followed by incubation with a silver salt solution.

30. The method of claim 28, wherein said providing a silver agent to the modified surface comprises coating the substrate with the modified surface with a polyethylene oxide hydrogel having a silver salt incorporated therein.

31. A method for making a modified surface on a substrate comprising:
coating the substrate with a photo-initiator;
incubating the photo-initiator-coated substrate in an aqueous monomer solution capable of free radical polymerization;
exposing the incubating substrate to ultraviolet light creating a modified surface on the substrate; and
rendering the modified surface anti-microbial.

32. A method for making a modified surface on a substrate comprising:
coating the substrate with a photo-initiator;
incubating the photo-initiator-coated substrate in an aqueous monomer solution capable of free radical polymerization;
exposing the incubating photo-initiator-coated substrate to ultraviolet light creating a modified surface on the substrate; and
ionizing the modified surface in an aqueous base solution so that the modified surface is negatively charged, followed by incubation with a silver salt solution.

33. A method for making a modified surface on a substrate comprising:
coating the substrate with a photo-initiator;
incubating the photo-initiator-coated substrate in an aqueous monomer solution capable of free radical polymerization;
exposing the incubating substrate to ultraviolet light creating a modified surface on the substrate; and
coating the substrate with the modified surface with a polyethylene oxide hydrogel having a silver salt incorporated therein.

34. A method for making a modified surface on a substrate comprising:
immersing a substrate in an alcoholic solution of a photo-initiator for a time sufficient for the photo-initiator to adhere to the surface of the substrate;
incubating the photo-initiator-coated substrate in an aqueous monomer solution capable of free radical polymerization;
subjecting the incubating substrate to ultraviolet (UV) light at a suitable wavelength to initiate polymerization of the monomer on the surface of the substrate, creating a modified surface on the substrate; and
rendering the modified surface anti-microbial.

35. The method of claim 34, wherein the UV wavelength is in the range of about 100 nm to about 400 nm.

36. The method of claim 34, wherein the UV wavelength is in the range of about 200 nm to about 400 nm.

37. The method of claim 34, wherein the UV wavelength is in the range of about 300 nm to about 400 nm.

38. An implantable medical device comprising a substrate with a modified surface prepared according to the method of claim 1.

39. The implantable medical device of claim 38, wherein the modified surface is hydrophilic.

40. The implantable medical device of claim 38, wherein the modified surface is bio-compatible.

41. The implantable medical device of claim 38, wherein the modified surface is anti-microbial.

42. An implantable medical device comprising a substrate with a modified surface prepared according to the method of claim 26.

43. The implantable medical device of claim 42, wherein the modified surface is hydrophilic.

44. The implantable medical device of claim 42, wherein the modified surface is polyacrylic acid.

45. The implantable medical device of claim 42, further comprising an anti-microbial agent on the modified surface.
